(19) United States
(12) Patent Application Publication
(10) Pub. No.: US 2006/0276970 A1
(43) Pub. Date: Dec. 7, 2006

(54) Apparatus and method for the ozone preservation of crops

(75) Inventors: Jeremy Barnes, Wark (GB); Ian Singleton, Whitley Bay (GB); Phil Harley, Hexham (GB); Nikos Tzortzakis, Chania (GR); Raveenia Roberts, Nassau (BS); Eva Tallentire, Northallerton (GB); Tahar Taybi, Newcastle-upon-Tyre (GB)

Correspondence Address: KINNEY & LANGE, P.A., THE KINNEY & LANGE BUILDING, 312 SOUTH THIRD STREET, MINNEAPOLIS, MN, 55415-1002, US

(73) Assignee: Biofresh Limited, Newcastle-upon-Tyne (GB)

(21) Appl. No.: 10/565,141

(22) PCT Filed: Jul. 19, 2004
(86) PCT No.: PCT/GB04/03102

§ 371 Date: Jun. 21, 2006

(30) Foreign Application Priority Data
Jul. 19, 2003 (GB) 0316970.3

(51) Int. Cl. C12Q 1/02; G06F 19/00

(52) U.S. Cl. 702/19; 435/29

(57) ABSTRACT

A method for substantially reducing the development of fungal spoilers of perishable produce such as fruit, salads and vegetables comprising the steps of arranging the products in a matrix in a substantially closed environment and introducing to the environment a gaseous mixture containing a prescribed concentration of ozone.

### FIELD OF THE INVENTION

The present invention relates the preservation of crops using ozone, and in particular to a method of preserving harvested crops using low concentrations of ozone and an apparatus for performing the method of the invention.

### BACKGROUND OF THE INVENTION

Post-harvest losses of fresh produce due to spoilage organisms are a significant problem world-wide for the horticultural and agricultural industries, resulting in reductions in both quantity and quality of marketable produce. Economic losses occur in every part of the supply chain from farm to supermarket. Spoilage in storage and transit is known in some cases to be as high as 30%, equivalent to a loss in revenue of £2 billion per annum to the United Kingdom industry. Current treatments, provide an unsatisfactory and unsustainable solution to the problem.

Ozone has long been recognised as a powerful anti-bacterial agent and has found widespread use in hospitals for sterilising implements, etc. It is also used extensively in the treatment of potable and waste water and for deodorising road transport containers to prevent the tainting of subsequent cargoes. There are examples here being that described in Russian patent RU2174316C2, and there are other examples in the scientific literature. It is apparent, however, from the latter, that attempts to reduce spoilage resulting from microbial activity are accompanied by visible lesions and other deleterious effects to the foodstuffs themselves. This is because of the relatively high concentrations of ozone that are generally applied, for instance, the Russian patent quoted above mentions concentrations of fifteen parts per million of ozone in air, which is almost three orders of magnitude greater in concentration than that prescribed hereunder, and is also seventy-five times greater than the acceptable U.K. limit for chronic human exposure.

Another method of treatment for foodstuffs is described in GB 2340376. In this patent application, a two-stage ozone treatment process is described. In part one of the process a foodstuff is exposed to an ozone concentration of between 0.01 to 10 ppm, for a period greater than 6 hours. In the second part of the process, the foodstuff is exposed to an ozone concentration of between 10 and 500 ppm for a period of 1 to 20 minutes. The problem with this method of treatment is that during the second part of the process the foodstuff is exposed to concentrations of ozone that are greater than permitted under food safety legislation in the United Kingdom.

Another method of treatment of foodstuffs is described in the published French patent application no 2603455. In this method a foodstuff is exposed to a gaseous mixture containing 0.5 ppm nitrogen oxide and 0.05 ppm ozone. It is claimed in FR 2603455 that the gaseous mixture reduces the production of ethylene.

U.S. Pat. No. 6,294,211 describes an apparatus and method of disinfecting a foodstuff contained in a vacuum using ozone in an amount between 0.1 ppm and 15% by weight. The patent indicates that the vacuum increases the efficacy of the ozone, thereby allowing a lower concentration to be used. Clearly, the requirement to contain the foodstuff in a vacuum makes the apparatus of this invention particularly costly.

U.S. Pat. No. 6,171,625 is concerned with the decontamination of animal feedstuffs and makes use of extremely high concentrations of ozone (10 to 20% by weight).

DE 4426648 describes a fumigation system using a minimum ozone concentration of 500 ppm, which is a massive concentration compared to that described in the present invention.

An ozone-enriched atmosphere is an attractive alternative treatment for inhibiting spoilage of horticultural produce. Although a highly reactive oxidant, ozone rapidly degrades to the normal molecular form of oxygen, so environmental disposal is not a problem. There is, however, a problem in that the ephemeral nature of the gas, makes the provision of a defined concentration, maintained for a lengthy period, and within the likes of a transit container, a crop store, or a food a handling operation, extremely difficult. In addition, its highly oxidising nature, can result in the produce receiving doses of ozone that cause serious damage to the crops themselves rendering them completely unsaleable, rather than merely inhibiting the process of microbial action.

The present invention relates to the finding that by maintaining a concentration of between fifty and two hundred parts per billion (one billion equals one thousand million i.e. 10⁹) by volume, of ozone in air the development of spoilage organisms is inhibited whilst the organoleptic characteristics of the crops themselves are not changed. Surprisingly, it has also been found that subjecting perishable horticultural produce to the above-mentioned concentrations of ozone (even for a relatively short period of time e.g. 2 hours) results in a preservative effect lasting significantly beyond the period of exposure. This novel finding indicates that low levels of ozone can stimulate endogenous defence mechanisms required to prevent microbial attack in produce stored and/or in transit (i.e. postharvest).

### SUMMARY OF THE INVENTION

One aspect of the invention provides a method of reducing the growth of microbial spoilage organisms in stored perishable commodities as specified in claim 1. Microbial spoilage organisms may include fungal spoilage organisms or bacterial spoilage organisms.

Another aspect of the invention provides a process for the vaccination of perishable products as specified in claim 11.

Another aspect of the invention provides an apparatus whose purpose is to generate ozone and distribute it through a matrix containing perishable products, in such a manner that the atmospheric concentration of ozone is maintained within the said prescribed concentration limits as specified in claim 15.

### BRIEF DESCRIPTION OF THE DRAWINGS

In the drawings, which illustrate preferred embodiments of apparatus according to one aspect of the invention, and are for exemplary purposes:

Diagram 1 is a schematic representation, not to scale, of a typical embodiment of the invention applied to a road transport container;

Diagram 2 is a schematic representation, not to scale, ot a typical embodiment of the invention applied to a crop store or warehouse; and

Diagram 3 is a schematic representation of a bag containing a perishable product.

Plate 1. Illustrates the extent of the effect of ozone enrichment on lesion development in tomato fruit maintained at 13° C./95% RH, 5-days after wound-inoculation with Botrytis cinerea. Control fruit were maintained for the same period in ‘clean’ (i.e. charcoal-filtered) air;

Plate 2a. Impact of a trace level of ozone enrichment on the development of Botrytis cinerea on easi-peel citrus and plums—spore suspensions containing 10³ (‘low’ inoculum concentration) Botrytis cinerea; and

Plate 2b. Impact of a trace level of ozone enrichment on the development of Botrytis cinerea on easi-peel citrus and plums—spore suspensions containing 10⁵ (‘medium’ inoculum concentration) of Botrytis cinerea.

### DETAILED DESCRIPTION OF THE PREFERRED EMBODIMENTS

A typical embodiment of the invention will now be described by way of example, and with the assistance of Diagram 1. A container 1, which in this example is a transport container of the type frequently used for road transport of perishable produce, is equipped with an ozone generator 2, which may be of the conventional type in which a corona, or silent, electrical discharge is taking place in a narrow gap is used to disassociate the natural oxygen in the air to subsequently re-combine as ozone, or, preferentially, is of the type described in International Publication number WO 00/14010 “Air Purification Device”. A fan 3, which may be integral to the container or ozone generator, or separate from it, blows the ozone laden air into/around the container, optionally with the assistance of appropriate distribution ducting 4. Because ozone is heavier than air, it is preferable to introduce it through ducting 4, attached to the roof of the container, so that it may descend through the produce to the floor, where a return duct 5, enables the air and any remaining ozone to be re-circulated.

In use, the container 1, is filled with perishable produce 6, loaded into crates 7, which are then stacked within it. In the exemplary embodiment three ozone sensors 8, are sited within the container. Such sensors are preferentially of the tungstic oxide semiconductor, as described in WO 95/35495, although other sensor technologies, for instance those based on ultra-violet absorption, may be utilised if appropriate. The sensors 8 are strategically positioned within the container to ensure that a representative concentration distribution may be measured and recorded by an electronic controller 9, so as to ensure that the prescribed ozone concentrations are maintained, but not exceeded, throughout the volume of the container. The electronic controller 9 receives the measurements made by the ozone sensors 8, and utilising this information, together with a pre-arranged protocol based on the physical parameters of the container 1, the prescribed concentrations of ozone, and optionally, the nature of the produce 6, contained therein, issues commands to the ozone generator 2 to moderate its production of the gas.

In similar embodiments, not illustrated, the ozone generation apparatus may be incorporated within air conditioning equipment, often in the form of refrigeration units as are commonly fitted to vehicle transportation units used for the conveyance of perishable foodstuffs. Likewise the arrangement is equally applicable to containers used in marine, air and rail transport.

A similar exemplary embodiment is described in Diagram 2. In this arrangement a large static store or warehouse 11, for produce 6, is equipped with an ozone generator 2, a plurality of ozone sensors 8, and one, or optionally, a plurality of electronic controllers 9. Such static stores are frequently equipped with apparatus to control the environment within the store, in particular temperature and humidity. Where such a system exists, or is planned, use is made of the existing fans and ducting associated with it to distribute ozone throughout the store/warehouse. Stores are often controlled in accordance with a model representative of gaseous fluid behaviour in the environment, such a model built into a computer program. Ozone may be released into the environment according to the concentration of ozone measured by the or each sensor, and the gaseous fluid behaviour model.

Diagram 3 illustrates a salad bag. The atmosphere within the bag has been charged with air containing a selected concentration of ozone in the range 50 to 200 ppb.

A further exemplary embodiment, not illustrated, is the application of the apparatus to food handling and packaging machinery, wherein the prescribed ozone environment is applied to perishable horticultural products whilst the product is being handled and packed.

One aspect of the invention provides a process of vaccination of perishable products against the post-harvest development of moulds and other fungal diseases. The process involves exposing the perishable products to an atmosphere containing a low concentration of ozone for a brief period.

Another aspect of the invention provides a method of reducing growth of microbial spoilage organisms in a stored perishable product.

The described vaccination effect results in a change in the expression of key genes related to spoilage as described in greater detail under paragraph 3.2 hereunder.

The experiments demonstrating how the process of vaccination and the method of storage work are described below in the section entitled, “Examples”:

### EXAMPLES

1. Mould/Disease Development is Suppressed in Produce Maintained in an Atmosphere Enriched with Trace Levels of Ozone

Tomatoes Infected with Botrytis.

Grey mould (Botrytis cinerea) lesion development was dramatically suppressed in tomato fruit exposed to an atmosphere containing 50 ppb ozone¹—even for a relatively short period (2-8 h). Fruit were wound-inoculated with a mycelial plug containing the pathogen² at day 0, transferred to charcoal-filtered air (CFA) or an ozone enriched CFA environment and then removed at intervals to ‘clean air’.
¹ Monitored using duplicate photometric analysers calibrated to US-EPA standards

² plugs (2.5 mm diameter) were removed from the advancing margins of 3-day-old B. cinerea cultures (4-5-day-old in the case of A. alternaria) and inserted into a superficial wound made in the surface of the fruit (two wound-inoculations per fruit)

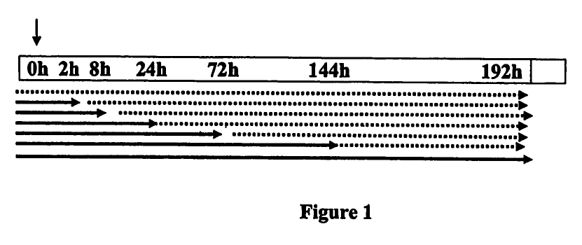

FIG. 1. Experimental design: Fruit maintained in ‘clean air’ (CFA, →) or ozone (50 ppb or 200 ppb (→) and then inoculated (↓) with an agar plug containing the pathogen. Exposure to ozone performed in the dark at 13° C. and 95%.

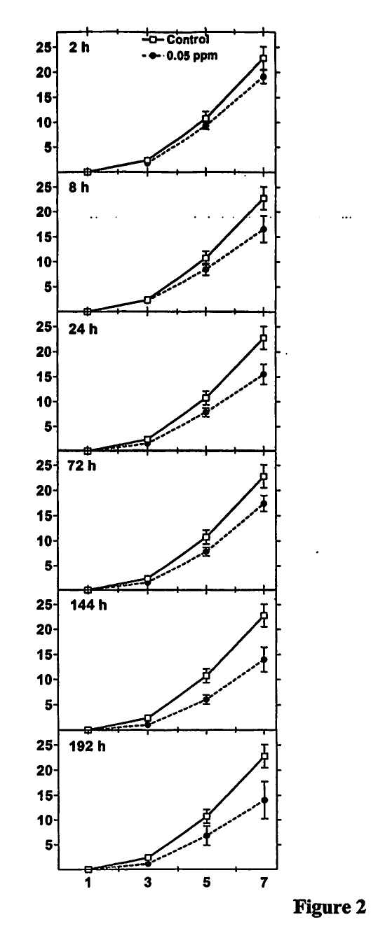

FIG. 2. Impacts of ozone-enrichment on the development of grey mould (Botrytis cinerea) on tomato (Lycopersicon esculentum L. cv. Mareta) fruit wound-inoculated with a mycelial plug (Lycopersicon esculentum L. cv. Mareta). Fruit were maintained in controlled environment chambers at 13° C. and 95% RH ventilated with charcoal-filtered ‘clean’ air (CFA, □) or CFA plus a trace level of ozone (50 ppb, ●). For experimental design see FIG. 1. Values represent mean (±SE) for 3-4 replicate fruit. Experiments repeated several times.

1.2. Tomatoes Infected with Alternaria.

The development of black spot (Alternaria alternata) was suppressed by more than 50% in fruit maintained in an atmosphere containing a trace level of ozone (50 ppb)—even for a relatively short period of time (2-8 h). Fruit were inoculated with a mycelial plug containing the pathogen and the experiment was performed according to design B (FIG. 1).

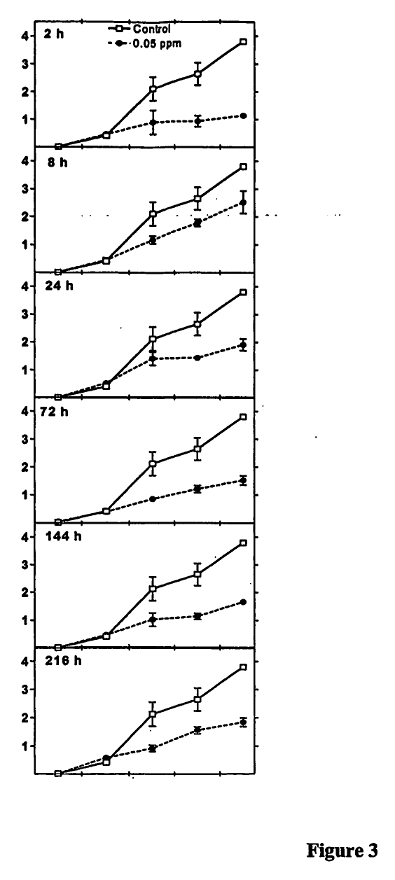

FIG. 3. Impacts of ozone-enrichment on the development of black spot (Alternaria alternata) raised on tomato fruit (Lycopersicon esculentum L. cv. Mareta) wound-inoculated with an agar plug containing mycelia of the pathogen. Fruit were maintained in controlled environment chambers at 13° C. and 95% RFL Chambers were ventilated with ‘clean’ (chracoal-filtered) air (CFA, □) or CFA plus a trace level of ozone (50 ppb, ●). Values represent mean (±SE) for 3-4 replicate fruit.

1.3 Tomatoes—Spore Production/Viability.

Using experimental design shown in FIG. 1. Fruit were wound-inoculated with a suspension containing c. 25×10³ spores of Botrytis, Alternaria or Colletotrichum, then incubated in clean air or in an atmosphere containing a trace level of ozone (50 ppb) at 13° C., 95% RH. After 9-12 d, spores were washed from inoculated fruit, counts made on a haemocytometer and recovered spore aliquots inoculated onto agar. In vitro spore germination was monitored following 72 h incubation in ‘clean air’ or ‘clean air’ plus a trace level of ozone. Exposure to an ozone-enriched atmosphere dramatically reduced the development (i.e. number of spores produced) by all pathogens. Moreover, exposure to a trace level of ozone also significantly reduced subsequent spore viability.

TABLE 1
Impact of trace ozone enrichment on fungal disease development
(spore production and viability).

| | | Clean Air (CFA) | 50 ppb O₃ |
|---|---|---|---|
| Botrytis cinerea | Spore prod. | 133.10 ± 8.740 | 7.50 ± 1.510 |
| Alternaria alternata | Spore prod. | 28.20 ± 1.639 | 5.83 ± 0.645 |
| Colletotrichum | Spore prod. | 9.29 ± 0.806 | 2.44 ± 0.526 |
| coccodes | | | |
| Botrytis cinerea | Spore germ. | 99.60 ± 0.163 | 96.76 ± 0.613 |
| Alternaria alternata | Spore germ. | 99.70 ± 0.153 | 99.86 ± 0.137 |
| Colletotrichum | Spore germ. | 99.00 ± 0.330 | 52.50 ± 4.550 |
| coccodes | | | |

1.4. Easi-Peel Citrus, Grapes & Plums—Spore Production.

Using experimental design shown in FIG. 1. Fruit were wound-inoculated with spore suspensions of Botrytis cinerea, then incubated in either clean air or an atmosphere containing a trace level of ozone (100 ppb) at 13° C. and 95% RH. Development of the pathogen (based on spore production) was dramatically reduced in fruit stored in an atmosphere containing a trace level of ozone.

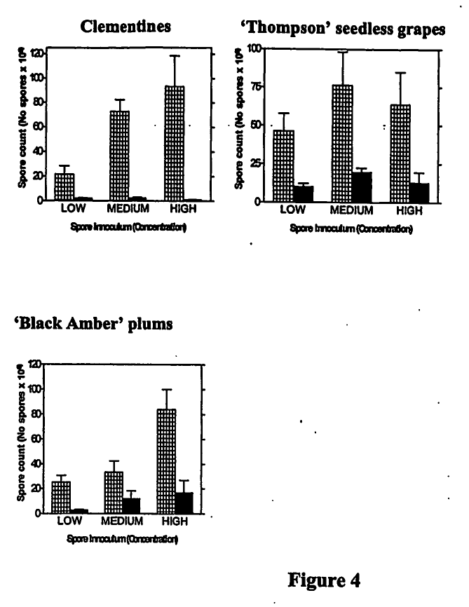

FIG. 4. Impact of a trace level of ozone enrichment (solid bars) on the development of Botrytis cinerea (based on spore counts) on easi-peel citrus, grapes & plums Fruit were wound-inoculated with spore suspensions containing 10³ (‘low’ inoculum concentration), 10⁵ (‘medium’ inoculum concentration) or 10⁷ (‘high’ inoculum concentration) Botrytis cinerea, then incubated in clean air or an atmosphere containing a trace level of ozone (100 ppb) at 13° C. and 95% RH, After 8-12 d, spores were washed from inoculated fruit, counts made on a haemocytometer and aliquots incubated on agar. Control fruit were maintained in ‘clean air’ (cross-hatch bars).

Plates 2a & 2b. Impact of a trace level of ozone enrichment on the development of Botrytis cinerea on easi-peel citrus and plums. Fruit were wound-inoculated with/in spore suspensions containing 10³ (‘low’ inoculum concentration), 10⁵ (‘medium’ inoculum concentration) or 10⁷ (‘high’ inoculum concentration) Botrytis cinerea, then incubated in clean air or an atmosphere containing a trace level of ozone (100 ppb) at 13° C. and 95% RR Control fruit were maintained in ‘clean air’.

1.5. Potatoes Infected with Silver Scurf (Helminthosporium solani).

Tubers cv. Estima were washed and selected for similar levels of silver scurf (Helminthosporium solani) infection. Prior to storage, tubers were sprayed with water in order to simulate a condensation event to promote the development of the disease. Tubers were then placed in chambers maintained at 3.5° C.±0.5° C., >95% RH) ventilated with either ‘clean air’ (charcoal-filtered air’; CFA) or ‘clean air’ plus a trace level of ozone (200 ppb). After four weeks, half the tubers were removed, then the temperature raised to 13±0.5° C. Eight weeks from the start of the experiment, tubers were removed from storage and groups of four and gently washed in 40 ml of water of which 10 ml of the resulting spore suspension was centrifuged at 1000 g for 10 minutes. The resulting pellet was resuspended in 1 ml of distilled water. A spore count was calculated for each tuber (n=10) using a haemocytometer.

Additionally, the initial and final surface area covered in silver scurf lesions was measured using a DELTA-T devices image analyser. Taking into account shrinkage of the potato over the storage period, the percentage of each tuber covered in silver scurf was calculated.

TABLE 2
Influence of ozone on the development (based on spore counts) of silver
scurf on potato tubers under simulated refrigerated storage conditions,
plus a rewarming period. Values bearing the same letter are not
significantly different at the 5% level of probability

Treatment

| Weeks 1-4 | Weeks 5-8 | Spores/ml |
|---|---|---|
| NFA | NFA | 4.9 × 10⁶ a |
| 200 ppb ozone | 200 ppb Ozone | 1.7 × 10⁶ b |
| 200 ppb ozone | NFA | 3.9 × 10⁶ c |

TABLE 3
Influence of ozone on the development (based on lesion area
development) of silver scurf on potato tubers under simulated
refrigerated storage conditions.

Treatment

| Weeks | Weeks | Lesion |
|---|---|---|
| 1-4 | 5-8 | development (%) |
| NFA | NFA | 29.79 a |
| 200 ppb ozone | 200 ppb ozone | 12.81 b |
| 200 ppb ozone | NFA | 21.34 a |

2. Produce Exposed to Trace Levels of Ozone are ‘Vaccinated’ Against Subsequent Infection

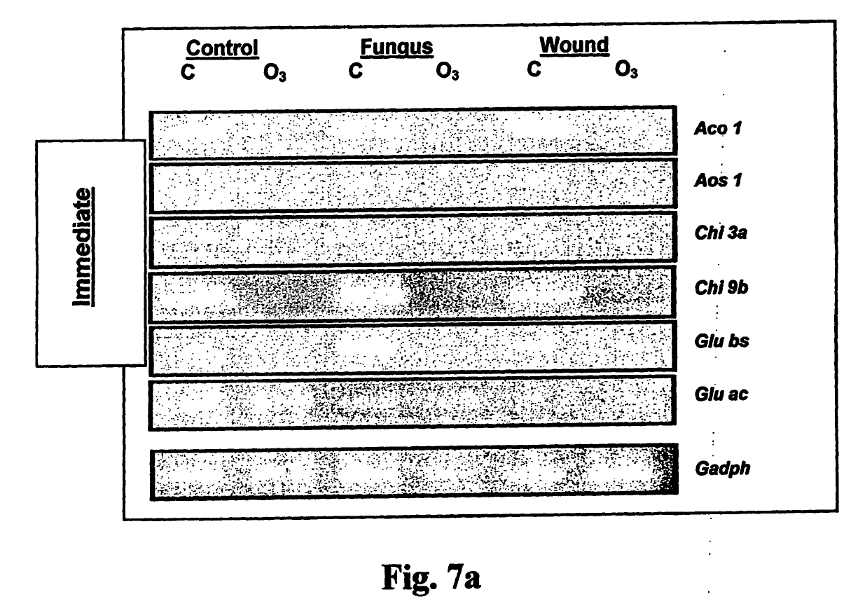

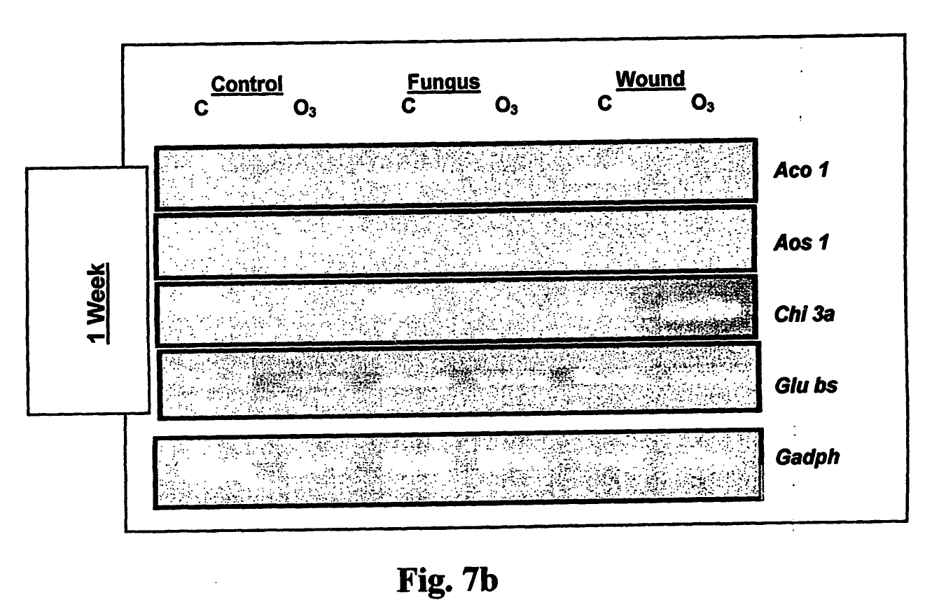

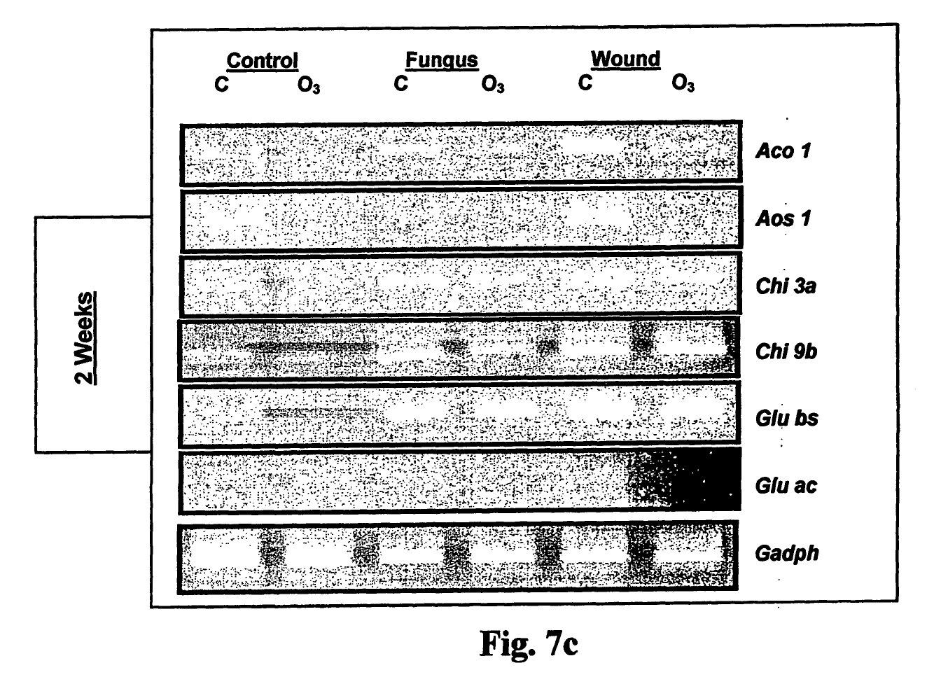

No direct effects of ozone (at concentrations upto 5.0 ppm) were observed on colony development in the targeted fungal pathogens during extensive investigations in vitro. This implies that the observed suppression of spoilage organisms by ozone results from molecular/biochemical changes in the treated produce per se—presumably through subtle shifts in the manner in which plant tissue responds to challenge by pathogens (see FIG. 7).

3. Exposure to a Trace Level of Ozone Effectively Vaccinates Tomato Fruit Against Subsequent Infection.

Tomato fruit were incubated for varying periods in ‘clean’ air or an atmosphere enriched with a trace level of ozone (50 ppb), then wound-inoculated with a mycelial plug of grey mould (Botrytis cinerea). Two experimental designs were adopted for the investigation of ‘memory’ effects induced by ozone:

(a) Fruit were incubated in clean air prior to transfer to an atmosphere containing a trace level of ozone in such a manner that fruit were wound-inoculated with the pathogen at the same physiological age. For gene expression studies, RNA was extracted from fruit snap-frozen in liquid nitrogen 24 h after wounding/inoculation, immediately following exposure to ‘clean air’ or ozone, and after 1 or 2 weeks' storage in ‘clean air’

Or, (b) Fruit were incubated in clean air or ozone and wound-inoculated with Botrytis cincerea either (i) immediately following the period of exposure, (ii) following 1 weeks' incubation in ‘clean air’ or (iii) following 2 weeks' incubation in ‘clean air’

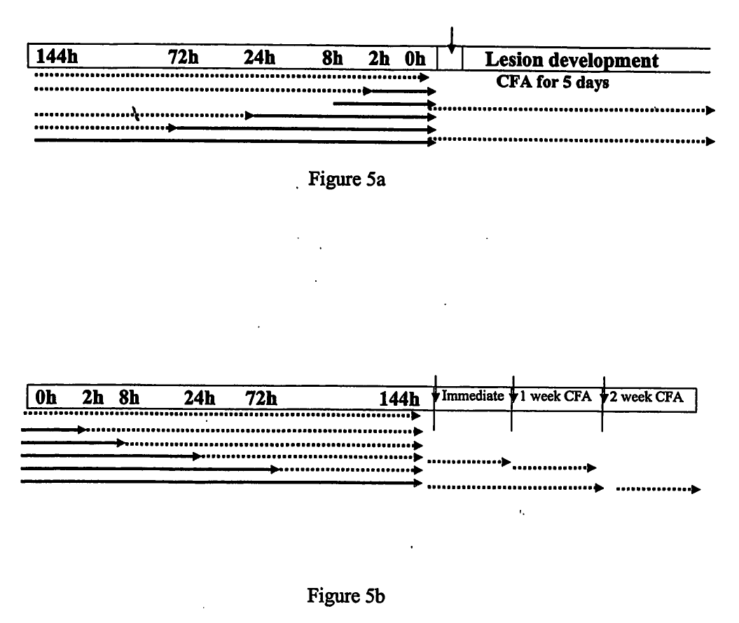

FIG. 5. Experimental designs to test whether exposure to a trace level of ozone vaccinates produce against subsequent infection. Tomato fruit were maintained in charcoal filtered air (CPA, →) or ozone (→) prior to wound-inoculation (↓) with grey mould (Botrytis cinerea). Tomato fruit were stored throughout in the dark at 13° C. and 95% RH. Lesion development was monitored during storage in clean air, over a 7-d incubation period.

Prior exposure of tomato fruit to an atmosphere containing a trace levels of ozone (even for a relatively short period e.g. 2-8 h) resulted in a marked suppression of pathogen development and this ‘vaccination effect’ persisted for up to two weeks after fruit were removed from the ozone-enriched atmosphere.

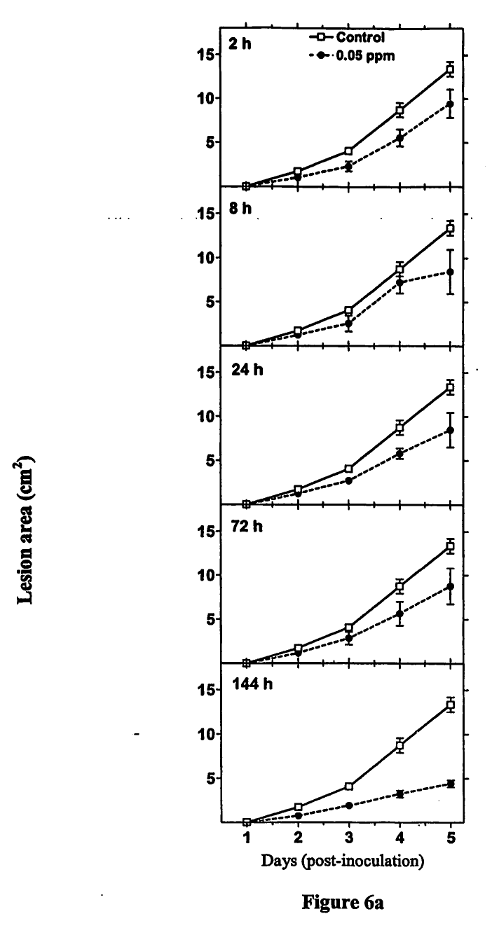

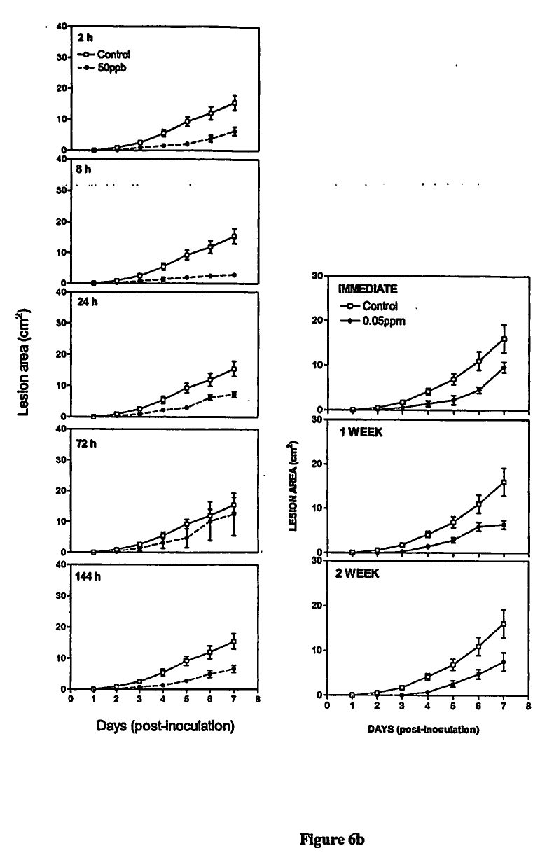

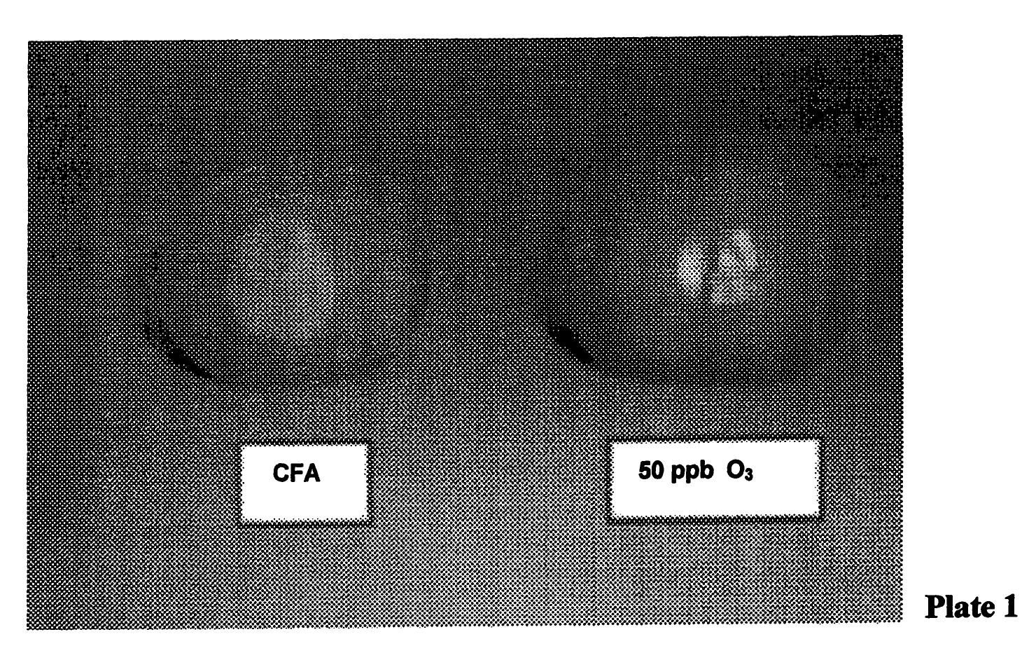

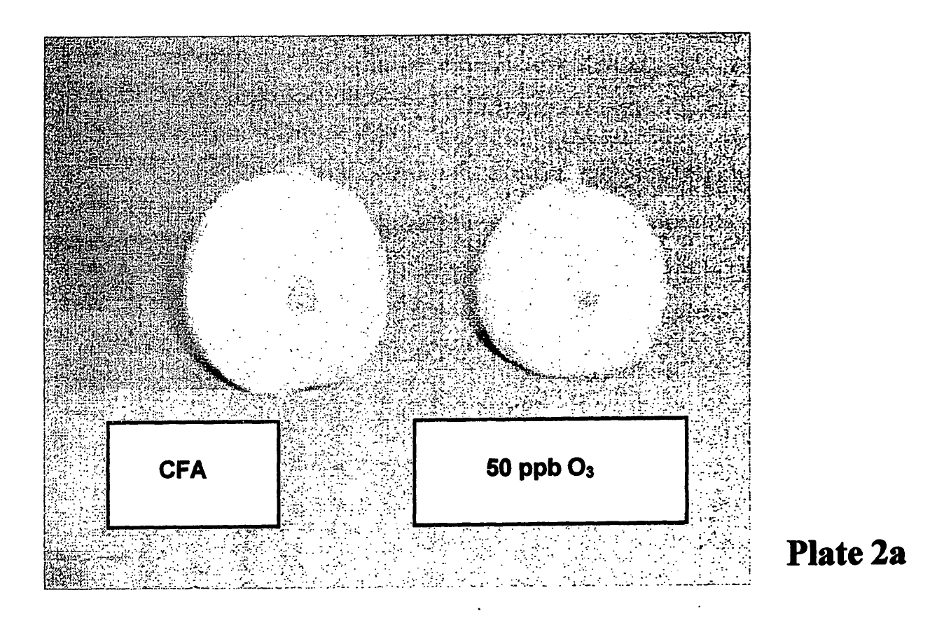

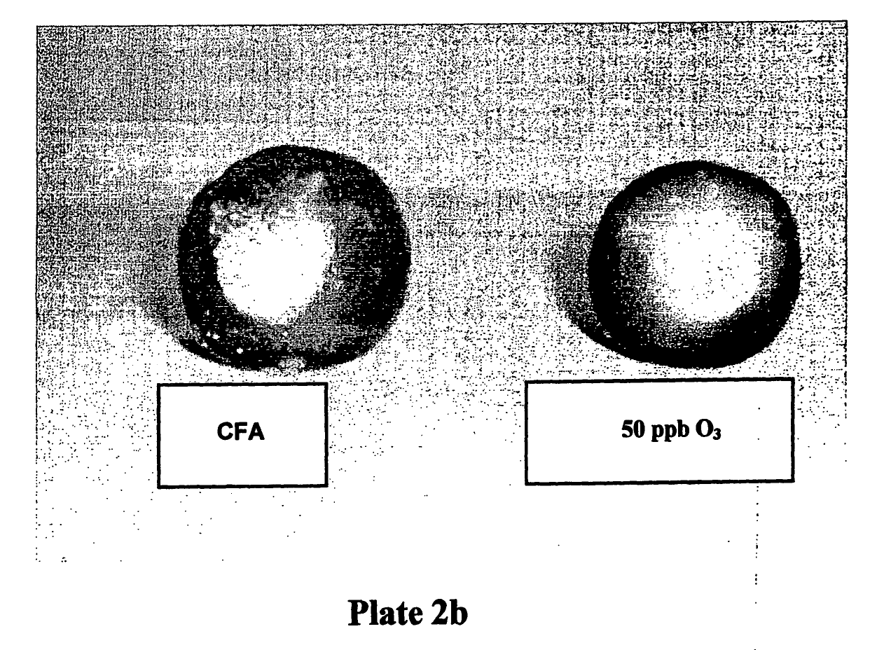

FIG. 6 Development of grey mould (Botrytis cinerea) on tomato fruit (Lycopersicon esculentum L.) previously exposed to ozone. Fruit were maintained at 13° C./95% RH in controlled environment chambers ventilated with ‘clean’ (chracoal-filtered) air (CFA, □) or CFA plus a trace level of ozone (50 ppb, ●). Experimental design (A) fruit were wound-inoculated at the same physiologically age immediately after exposure to ozone (B) Fruit were exposed to ozone for 144 h, wound-inoculated immediately with Botrytis, or transferred to CFA for 1 or 2 weeks prior to wound-inoculation. Fruit were inoculated with a plug containing mycelia of Botrytis cinerea, and incubated in duplicate controlled environment chambers receiving clean air (‘immediate inoculation treatment’). Values represent mean (±SE) lesion development for replicate batches of fruit (3-4 fruit per batch).

3.2. Mechanism(s) Underlying Vaccination Effect Induced by Exposure to a Trace Level of Ozone.

A decline in the expression of key genes involved in signal-transduction (e.g. Aco1 (aminocyclopropancarboxylic acid oxidase—a key enzyme mediating ethylene biosynthesis) and Aos (allene-oxide synthase—a key enzyme governing jasmonate synthesis)), as well as defence against biotic/abiotic stresses (e.g. Chit3α (chi3-type acidic chitinase), Chit9b (chi9-type basic chitinase), Glucac (acidic β-1-3 glucanase), Glucbs (basic β-1-3 glucanase) and Hpl (hydroperoxide lyase), was detected in tomato fruit exposed to 50 ppb ozone and effects persisted for upto two weeks' following transfer of fruit to ‘clean air’. Shifts in gene expression patterns were therefore consistent with the observed effects of trace ozone-enrichment on the development of a variety of fungal pathogens.

FIG. 7. Ozone-induced suppression of gene expression (probed by RT-PCR) induced by wounding/pathogen. Aco1 (aminocyclopropancarboxylic acid oxidase) and Aos (allene-oxide synthase) govern the production of ethylene and jasmonate, respectively. These are key signalling molecules. Chit3a (chi3 type chitinase acidic), Chit9b (chi9-type chitinase basic), Glucac (β-1-3 glucanase acidic), Glucbs (β-1-3 glucanase basic), and Hpl hydroperoxide lyase) are all involved with defence against pathogens and other stresses. Gapdh was used as the control gene. Measurements made on tomato fruit (Lycopersicon esculentum L.) incubated throughout in controlled environment chambers maintained at 13° C. and 95% RH and ventilated with clean air or 50 ppb ozone. FIG. 7a, immediate; FIG. 7b, 1 week; FIG. 7c 2 weeks.

1-30. (canceled)

31. A method of stimulating the endogenous defense mechanisms of a perishable product against microbial attack for a period of effective defense comprising the steps of:
a) arranging perishable products in a matrix within a substantially closed environment;
b) exposing the perishable products to a gaseous mixture of air and ozone at a prescribed concentration for a period of exposure, the prescribed ozone concentration being a selected concentration in the range of around fifty to five hundred parts per billion (ppb) by volume in air; and
c) removing the products from the substantially closed environment after the period of exposure;
wherein the period of effective defense against microbial attack resulting from ozone exposure during the period of exposure is between two and five hundred hours.

32. A method as claimed in claim 31, wherein the period of exposure resulting in effective defense against microbial attack of perishable produce is between around two and around five hundred hours.

33. A method as claimed in claim 31, wherein the period of effective defense is between two and eight hours.

34. A method as claimed in claim 31, wherein the prescribed concentration is in the range of around fifty to around two hundred ppb by volume.

35. A method as claimed in claim 34, wherein the prescribed concentration is in the range of around fifty to around one hundred ppb by volume.

36. A method according to claim 31, wherein the prescribed concentration is fifty or one hundred or two hundred or five hundred ppb by volume.

37. A method according to claim 31, comprising the further step of maintaining the relative humidity in the enclosed environment at around ninety five percent.

38. A method according to claim 31, comprising the further step of maintaining the temperature in the enclosed environment at four to thirteen degrees C.

39. A method according to claim 31, wherein the substantially closed environment consists of a crop store, a warehouse, or a freight transport container.

40. Apparatus for performing the method of claim 1, comprising an ozone generator, an ozone sensor and a controller, wherein generated ozone is released into the environment until the prescribed ozone concentration is reached, and wherein the ozone sensor measures the concentration of ozone in the environment, and when the measured concentration of ozone falls below the prescribed ozone concentration the controller commands the ozone generator to release ozone into the environment, so as to maintain continuously the ozone concentration in the environment substantially at the prescribed concentration.

41. Apparatus according to claim 40, wherein ozone is released into the environment by the ozone generator via a plurality of inlets.

42. Apparatus according to claim 40, comprising a plurality of ozone sensors.

43. Apparatus according to claim 40, wherein the controller includes computer software, the software including a model representative of the of gaseous fluid behavior in the environment, and wherein ozone is released into the environment according to the concentration of ozone measured by the or each sensor, and the gaseous fluid behavior model.
